(19) United States
(12) Patent Application Publication
(10) Pub. No.: US 2015/0320586 A1
(43) Pub. Date: Nov. 12, 2015

(54) CONDOM HAVING A FOAM TIP

(71) Applicant: Ansell Limited, Richmond (AU)

(72) Inventors: KC Nguyen, Dothan, AL (US); Chayapon Ngowprasert, Phunphin Surat Thani (TH); Chintana Netrung, Phunphin Surat Thani (TH); Chayaporn Pongthanomsak, Phunphin Surat Thani (TH)

(21) Appl. No.: 14/703,043

(22) Filed: May 4, 2015

Related U.S. Application Data
(60) Provisional application No. 61/989,711, filed on May 7, 2014.

(51) Int. Cl. A61F 6/04; B29C 41/24; B29C 41/14

(52) U.S. Cl. CPC A61F 6/04; B29C 41/14; B29C 41/24; A61F 2006/044; B29L 2009/005

(57) ABSTRACT

A condom that includes a tubular shaft comprising a polymeric composition and having an open end and a closed end, a hollow interior chamber disposed between the open end and the closed end, and an open-cell foam tip portion disposed on at least one of an internal portion of the closed end and/or an exterior surface of the closed end, wherein the tubular shaft and the open end are adapted for receiving a penis of a wearer.

### CROSS-REFERENCE TO RELATED APPLICATIONS

This application claims the benefit of priority to U.S. Provisional Application No. 61/989,711, filed May 7, 2014, which is herein incorporated in its entirety.

### BACKGROUND

1. Field of the Invention

Embodiments of the present invention generally relate to condoms and, more particularly, to condoms comprising at least one absorbent layer and methods of fabricating condoms.

2. Description of the Related Art

Condoms and other prophylactics comprise an elastomeric layer in a generally tubular shape, with an open end and a closed end having a tip portion, to provide physical barriers against the transmission of bodily and other fluids. Condoms sometimes comprise a lubricant or a spermicide located therein. However, lubricants and spermicides spread out into all areas of condoms. In the case of lubricants, this can have the effect of desensitizing the user, which can be a problem especially for users having erectile dysfunction. In the case of spermicides, the spermicides may have decreased efficacy to kill the sperm in semen if the spermicide spreads out. Furthermore, if additional spermicide is added to a condom, it may exacerbate erectile dysfunction as spermicides in liquid form are lubricious. And, unfavorable side-effects are associated with spermicides, such as a higher incidence of skin lesions, which can become avenues for infections.

Therefore, a condom providing enhanced protection against the transmission of bodily fluids and other fluids, while imparting enhanced stimulation during sexual activity, while delivering/localizing active ingredients so that less active ingredient may be used, represents an advance in the art.

### SUMMARY

Embodiments according to the invention include condoms having a foam tip capable of absorbing semen as well as delivering/localizing one or more active ingredients to a male wearer, and methods for manufacturing such condoms, substantially as shown in and/or described in connection with at least one of the figures, as set forth more completely in the claims, are disclosed. Various advantages, aspects, and novel features of the present disclosure will be more fully understood from the following description and drawings.

The foregoing summary is not intended, and should not be contemplated, to describe each embodiment or every implementation of the present invention. The Detailed Description and exemplary embodiments therein more particularly exemplify the present invention.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments. It is to be understood that elements and features of one embodiment may be in other embodiments without further recitation. It is further understood that, where possible, identical reference numerals have been used to indicate comparable elements that are common to the figures.

FIG. 1 depicts a perspective view of a condom in accordance with embodiments of the invention;

FIG. 2 depicts a cross section of the condom of FIG. 1 taken along line 2-2 in accordance with embodiments of the invention;

FIG. 3 depicts an alternative cross section of the cross section of a condom of FIG. 2 taken along line 3-3 in accordance with embodiments of the invention;

FIG. 4 depicts a cross section of the cross section of a condom of FIG. 2 taken along line 4-4 in accordance with embodiments of the invention;

FIG. 5 depicts a flow diagram of a method for manufacturing a condom in accordance with one or more embodiments of the invention;

FIG. 6 depicts a flow diagram of at least one exemplary method for manufacturing a condom in accordance with one or more embodiments of the invention; and

FIG. 7 depicts a flow diagram of at least one exemplary method for manufacturing a condom according to one or more embodiments of the invention.

### DETAILED DESCRIPTION

Embodiments according to the present invention pertain to condoms comprising a tubular polymeric layer having a foamed polymeric material disposed thereon that is adapted to localize active ingredients and/or semen. Exemplary embodiments of the invention comprise a condom having a single layer, including a tip portion comprising a polymeric foam. The tip portion comprises an open-cell foamed tip portion formed on at least one of an inner surface or an exterior surface of the tip portion.

The tip portion, comprising the open-cell polymeric, foam is adapted to perform at least one of several functions. One function is to localize active ingredients in the tip. For example, spermicide can be located within the foamed tip. Alternatively, a lubricant and/or a spermicide can be located in the foamed tip. Furthermore, a vasodilator, e.g., a discrete amount of vasodilator, with or without a lubricant and/or spermicide, is localized within the foamed tip, providing an effective dose at the penis tip to enhance erections, which is typically where a vasodilator is needed. The foamed tip is also adapted to absorb semen after ejaculation, trapping semen so that if the condom breaks, sperm is less likely to impregnate a female. Furthermore, the foamed tip comprises a textured surface as compared with the tip of non-foamed layer, which provides stimulation and pleasure during intercourse. Moreover, exemplary embodiments of a condom comprising a foamed tip on an exterior surface are adapted to provide the same functions to the partner of the male wearing the condom.

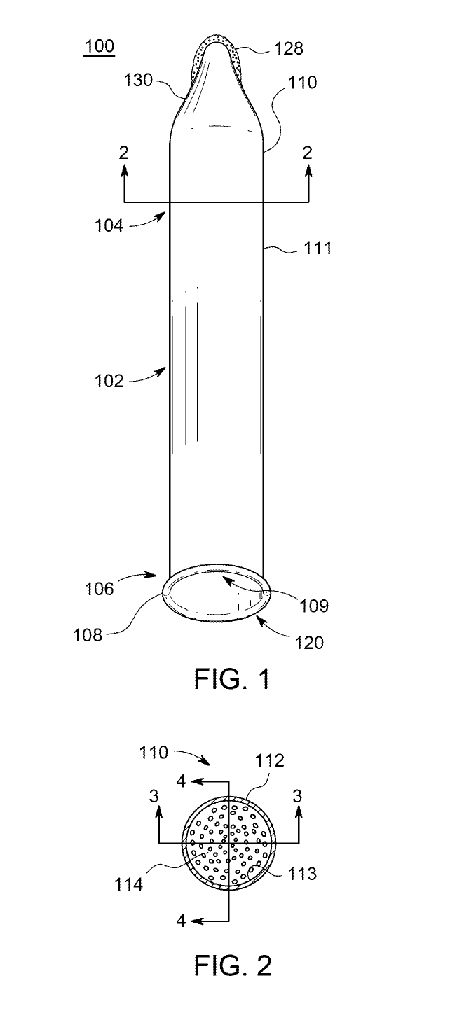

FIG. 1 depicts a perspective view of a condom 100 in accordance with embodiments of the invention. The condom 100 comprises a tubular shaft 102 having a closed end 104 and a back end 106, which is open-ended for receiving a penis at opening 109 and optionally comprises ring 108. An outer or exterior shaft surface 111 is disposed between the closed end 104 and the back end 106. The condom 100 further comprises a tip portion 110 adjacent to the closed end 104. A hollow interior chamber 120 for receiving a penis is disposed inside the condom 100, between the opening 109 and the tip portion 110. The condom 100 is a typical length of condoms, for example, approximately 180-220 mm. The cross-sectional thickness of the condom 100 is also a typical thickness, for example, 0.04-0.12 mm. The condom 100 typically comprises a circumference of approximately 100-120 mm, and may neck down from the back end 106 to the closed end 104 and tip portion 110. Embodiments according to the invention include an outer foamed tip portion 128 that is disposed on an exterior surface 130 of the condom 100 and/or a foamed tip portion formed on an inner or interior surface (not shown) of the hollow interior chamber.

FIG. 2 depicts a cross section of the condom 100 of FIG. 1 taken along line 2-2 in accordance with embodiments of the invention. The tip portion 110 comprises an inner surface 113. The inner surface 113 meets a foamed interior tip portion 114 as is more fully described below. The foamed tip portion 114 comprises a foamed polymeric composition, as discussed above, and may comprise a similar polymeric composition for the tubular shaft 102. Optionally, the polymeric composition for the foamed interior tip portion 114 could be made from different materials, or combinations thereof, that are compatible with the polymeric composition of the tubular shaft 102. The tip portion 110 is approximately 20-60 mm in length.

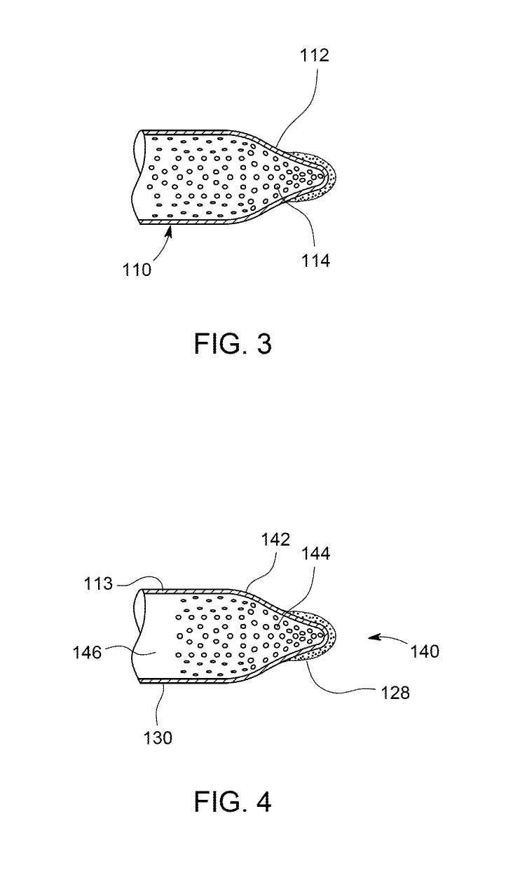

FIG. 3 depicts an alternative cross section of the cross section of a condom 100 of FIG. 2 taken along line 3-3 in accordance with embodiments of the invention. The alternative cross section has no foamed tip disposed on the outer surface 130 of the tip portion 110. As discussed above, the tip portion 110 comprises a foamed interior tip portion 114 and an unfoamed outer tip portion 112. The foamed interior tip portion 114 contacts the wearer of the condom. Also, the foamed interior tip portion 114 may be disposed only on or adjacent to a distal end of the tip portion 110 and thereby contact the very end of a wearer's penis, for example, only the foamed interior tip portion 114 is foamed, which may extend 20-60 mm from the closed end toward the open end.

FIG. 4 depicts a cross section of the cross section of a condom of FIG. 2 taken along line 4-4 in accordance with embodiments of the invention. The alternative The cross section depicts embodiments according to the invention in which the alternative foamed interior tip portion 144 extends further from the closed end 104 (not shown) to the back end 106 (not shown). In other words, in some embodiments according to the invention, the tip portion 140 may, for example, surround the entire tip of a penis while, in other embodiments, only a front aspect of a penis may be contacted by the tip portion 140. A longer foamed interior tip portion 144 provides more foam for absorbing semen, and/or providing more surface area that can contact a penis, enhancing stimulation and/or providing more surface area in which a lubricant or vasodilator can be contained therein. As shown, the foamed tip portion 144 is adjacent to an unfoamed portion 146 on inner surface 113. In some embodiments according to the invention, the tip portion 140 is adapted to match, approximately, the retroglandular sulcus of a wearer's penis, which is a particularly sensitive area on a male. In some embodiments, the tip portion 140 comprises a length and shape adapted to fit over the entire glans of a penis. Any or all of the foam disposed on condoms herein may comprise a thickness of approximately 20 to approximately 100 microns. An external portion of a tip portion 140 comprises a portion 142 of the outer surface 130, which is not foamed and wherein the outer surface 130 comprises an optional outer foamed tip 128.

At least one exemplary embodiment according to the invention includes a condom having at least one active ingredient displaced in the foam tip. Active ingredients comprise medicines, such as anti-virals, anti-microbials, and/or anti-fungals, and vasodilators such as niacin, sildenafil citrate, and/or nitroglycerin and/or male desensitizing agents such as benzocaine, and warming liquids and/or gels, such as caffeine or menthol, and/or the like, and/or spermicides such as nonoxynol-9. Because active ingredients may be disposed within the foam tip, less of the active ingredient(s) is needed because the active ingredient(s) remains localized and a higher concentration is therefore delivered to the tip of the penis, where it is needed, during or before sex. For example, typically, benzocaine is administered in a 3.0 to 7.5% in a water-soluble base medium. (21 CFR §348.10) Therefore, a typical dosage within a condom is 300-400 mg at 4.5% concentration. Because the majority of the agent, benzocaine, is localized at the foam tip, less dosage is needed, e.g., 200-300 mg to provide efficacy.

In at least one exemplary embodiment, the foam tip releases the at least one active ingredient because of the pressure, and/or heat and/or friction generated during sex. A lubricant may also be disclosed in the foam tip irrespective of whether an active ingredient(s) is disposed therein, providing multi-functional condoms.

Methods for forming condoms in accordance with embodiments of the present invention are also included herein. In one or more embodiments, at least one method includes providing a former comprising an axial length, a circumference, and a plurality of depressions, ribs, or protrusions disposed along at least a portion of the length and around or along the circumference of the former as is disclosed in commonly assigned U.S. patent application Ser. No. 13/243,038, which is herein incorporated by reference in its entirety. The method of one or more embodiments includes disposing a polymeric layer on the former, generally by a dipping process, to form a polymeric layer on the former, allowing the polymeric layer to dry, and dipping the former having the polymeric layer disposed thereon into a foamed polymeric composition thereby disposing a foamed layer onto the polymeric layer, and curing the polymeric layer to form a cured polymeric condom having a foamed tip.

The former may be a smooth former or, alternatively, a former having depressions on the surface, e.g., a textured former, which create ribs, studs, and the like, on an interior surface of a condom. In one or more embodiments, the former may include a tubular body having a first end and a second end. The tubular body may have an overall shape that is similar to the shape of a penis, thereby resulting in the polymeric layer of the condom described above. The tubular body of the former may include a base segment that is disposed adjacent to the first end and extends from the first end toward the second end. In one or more embodiments, the second end is utilized to form a closed end of the condom described above, while the first end of the former is utilized to form an open end and a base portion of the condom described above. The tubular body of the former according to one or more embodiments may also include a middle portion that extends from the base portion toward a tip portion, which is disposed adjacent to the second end.

In one or more embodiments, the step of disposing a polymeric layer on the former includes disposing a coagulant component on the former, as is known to those of skill in the art, and dipping the coagulant coated former into a bath or tank containing a polymeric composition as described herein. In one or more embodiments, the former may be dipped in a bath or tank containing a polymeric composition without first disposing a coagulant component on the former. Other methods of disposing a polymeric layer on the former may be utilized, such as spraying or solvent dipping. Additionally, the temperature of the polymeric composition may be controlled, for example, the temperature may be from approximately 20° C. to approximately 30° C. during the dipping process. Also, embodiments of the polymeric compositions according to the invention comprise additives to control or modify the properties of the elastomeric composition, such as the viscosity of the composition as well as the physical properties, for example, lubricity, tensile strength, puncture resistance, and the like, of condoms formed therefrom. The viscosity of the polymeric compositions according to embodiments of the invention is, for example, approximately 20 to approximately 60 centipoises.

The polymeric composition of one or more embodiments may also include a cure package or vulcanization agents to promote cross-linking during the curing process. As the former is dipped into a bath or tank containing a polymeric composition, the dwell time and immersion and extraction speeds of the dipping process may be controlled and modified to adjust the thickness of the resulting layer that forms the polymeric layer. In one or more embodiments, the polymeric composition disposed on the former is cured or otherwise treated to form a cured polymeric layer. In one or more embodiments, the polymeric layer is dried in ambient air and heated to a temperature ranging from about 40° C. to about 150° C. The former may be formed from glass, borosilicates, ceramic materials, metallic materials, and/or other materials known in the art.

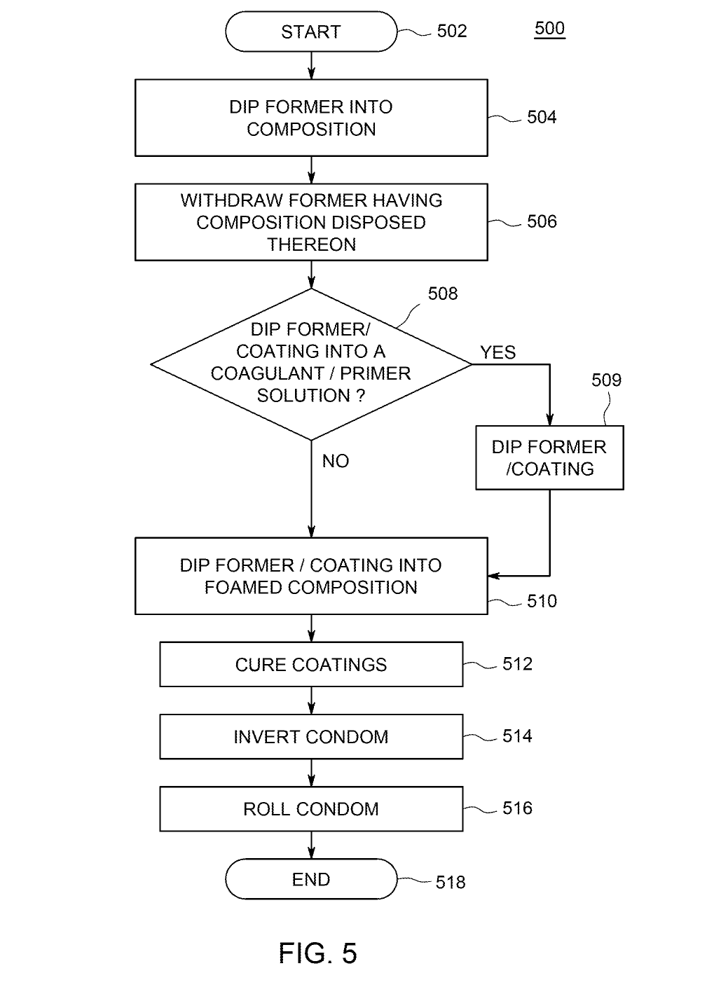

FIG. 5 depicts a flow diagram of a method 500 for manufacturing a condom in accordance with one or more embodiments of the invention. In some embodiments, each step of the method 500 is performed. In some embodiments, some steps are omitted and/or additional steps are performed. Also, in some embodiments, steps may be implemented in different sequences. For example, any method herein comprises optional steps for cleaning a former, for example, with brushes, such as nylon brushes, and/or pre-heating the former, for example, with hot air or within an oven at a temperature of approximately 35-60° C. Also, in at least one or more embodiments, a coagulant component or solution, as discussed below, may be disposed onto the former. If a coagulant solution is disposed on the former, it is optionally dried, for example, at 60-70° C. for approximately 2-5 minutes.

A method 500 starts at step 502 and proceeds to step 504, at which point a former is dipped into an unfoamed polymeric composition as are described herein, which comprise vulcanizing agents, activators, accelerators, antioxidants, and, optionally, stabilizers, thixotropic agents and/or the like as are known to those in the art. At least one exemplary embodiment of a formula for a polymeric composition according to embodiments of the invention is shown in Table 1. The formula may comprise additional components, such as potassium hydroxide, ammonium solutions, and/or the like for diluting the polymeric composition, adjusting the pH, and the like. The polymeric composition may contain additional surfactants, such as a polysorbate, for example, TWEEN® 20, to stabilize the foam. The formulation in Table 1 may be used for foamed and unfoamed polymeric compositions, subtracting, for example, foam stabilizers, as appropriate.

TABLE 1

| Component | Amount (PHR) | Function |
|---|---|---|
| Polymeric resin/Latex | 100 | Base polymer |
| Sulphur | 0.40-1.50 | Vulcanizing agent |
| Zinc Oxide | 0.00-1.00 | Activator |
| Zinc Diethyldithiocarbamate | 0.25-1.00 | Accelerator |
| Sodium Dibutyldithiocarbamate | 0.10-0.50 | Accelerator |
| Butylated reaction product of p-cresol | 0.00-1.00 | Antioxidant |
| and/or dicyclopentadiene | | |
| Polyoxyethylene Cetyl-Stearyl ether | 0.00-2.00 | Latex & Foam |
| | | Stabilizer |
| Octanoic acid and/or Potassium salt | 0.00-0.50 | Stabilizer |
| Methylhydroxypropyl cellulose | 0.00-0.50 | Foam Thickener |
| Polyacrylate | 0.00-0.50 | Foam Thickener |

The method 500 proceeds to step 506, wherein the former is removed from the polymeric composition, thereby forming a polymeric layer, i.e., a first coating of the polymeric composition on the former. After the former is removed from the unfoamed polymeric composition, forming a polymeric layer on the former, it is optionally partially dried, for example, at 80-100° C. for approximately 3-5 minutes, although the polymeric layer is gelled but not cured at this point. At step 508, a decision is made whether to apply a coagulant or primer to the first coating. If the answer is yes, the method 500 proceeds to step 509 and the former having the first coating is dipped into a coagulant or primer solution, as more fully described below, and dried at, for example, 60-70° C. for approximately 3 minutes and then proceeds to step 510. If the answer is no, the method 500 proceeds directly to step 510, at which point the first coating is dipped into a foamed polymeric composition. The former is dipped, for example, for a few to several seconds, into the foamed polymeric composition as far as necessary as to form a foamed polymeric coating, e.g., a foamed tip, onto the first coating. Because the foamed polymeric composition is disposed on the first coating before the first coating undergoes a curing step (discussed below), the foamed tip and the first coating are integrally formed with each other. In this context, integrally formed means that the foamed tip and the first coating cannot be separated without destroying one or both. Furthermore, the foamed tip and the first coating are adhered to each other without the use of, for example, an adhesive layer. A primer, discussed above, may be used to improve the adherence of the foamed tip to the first coating.

For example, the former having the first polymeric coating may be dipped 20 mm deep into a bath containing the foamed polymeric composition to form a foamed tip. In some embodiments, the former having the first polymeric coating disposed thereon may be dipped, for example, 60 mm deep into the foamed polymeric composition bath. The former having both the first polymeric coating and the foamed polymeric coating is then removed from the bath of foamed polymeric composition and the method 500 proceeds to step 512, at which point the first polymeric coating and the foamed polymeric coating are cured, as discussed above and may be washed, leached, etc., as is known to those in the art. The method 500 then proceeds to step 514, at which point the condom is inverted and rolled at step 516. At step 518, the method 500 ends. In at least one embodiment according to the invention, the inverting and rolling steps are performed by a revert-stripping process, i.e., the condom is stripped and inverted in the same step. For example, a water jet, such as at 2-4 bars of pressure, sprays the condom and strips and inverts the condom at the same time. Optionally, the condom may be stripped partially from the former and a roller used to remove and invert the condom from the former. Other processes include using compressed air to invert the condom onto tubing and then rolling the condom.

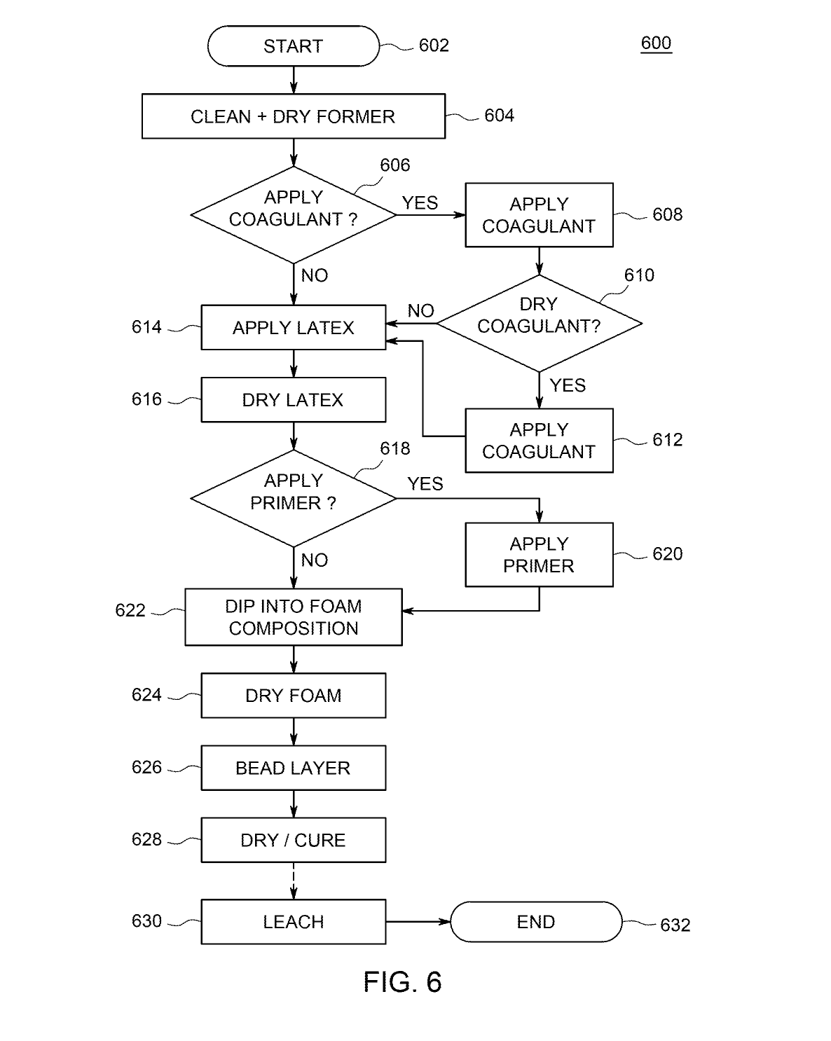

FIG. 6 depicts a flow diagram of at least one exemplary method 600 for manufacturing a condom in accordance with one or more embodiments of the invention. In some embodiments, each and every step of the method 600 is performed. In some embodiments, some steps are omitted or additional steps are performed. Also, in some embodiments, steps may be implemented in different sequences. A method 600 starts at step 602 and proceeds to step 604 at which point a former is cleaned and dried. At step 606, a decision is made whether to apply a coagulant to the former. If the answer is no, the method proceeds to step 614, at which point a latex or polymeric composition is applied. If the answer is yes, the method 600 proceeds to step 608, where a coagulant is applied, such as a powder or coagulant solution. A decision is then made whether to dry the coagulant solution at step 610. If the answer is no, the method 600 proceeds to step 614. If the answer is yes, the method 600 proceeds to step 612 where the coagulant solution is dried at, for example, 65° C. for approximately 2-5 minutes, and then the method 600 proceeds to step 614, as above.

The method 600 next proceeds to step 616, wherein the former is removed from the polymeric composition and dried at, for example, 70-110° C. for approximately 2-5 minutes, thereby forming a first coating of the polymeric composition on the former. At step 618, a decision is made whether to apply a primer, as discussed more fully below, to the first polymeric coating. If the answer is yes, the method 600 proceeds to step 620 and the former having the first polymeric coating is dipped into a primer and/or coagulant solution. A primer may be used to modify the surface chemistry to promote bonding between, for example, a first polymeric coating and a second polymeric coating. A coagulant may be used to de-stabilize the polymeric composition, promoting a gelling of the polymeric composition. In some embodiments, the former having the first polymeric coating is dipped into the primer solution, for example, one inch deeper than it will be dipped into the foamed composition described below. If the answer is no, the method 600 proceeds directly to step 622, at which point the first polymeric coating is dipped into a bath of foamed polymeric composition for a few seconds, wherein a foamed coating is adhered to the first polymeric coating. The former having both the first polymeric coating and the foamed polymeric coating is then removed from the bath of foamed polymeric composition and the method 500 proceeds to step 624, at which point the foamed polymeric coating is optionally dried at, for example, 65° C. for approximately 2-5 minutes. At step 626, a beading step is performed. At step 628, the first polymeric coating and the foamed coating are cured at, for example, 90-120° C. for approximately 3-5 minutes, to form a condom. At step 630, the condom is optionally leached in hot water. At step 632, the method 600 ends.

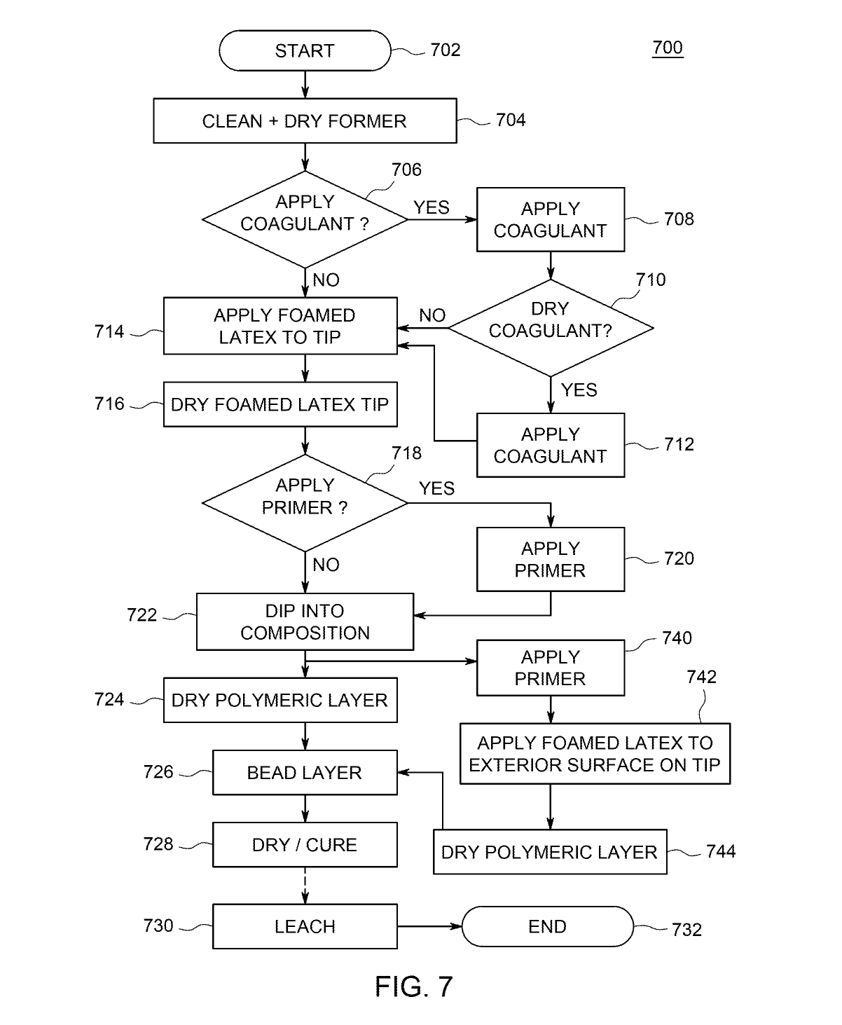

FIG. 7 depicts a flow diagram of at least one exemplary method 700 for manufacturing a condom according to one or more embodiments of the invention. In some embodiments, each and every step of the method 700 is performed. In some embodiments, some steps are omitted or additional steps are performed. Also, in some embodiments, steps may be implemented in different sequences.

A method 700 starts at step 702 and proceeds to step 704 at which point a former is cleaned and dried. At step 706, a decision is made whether to apply a coagulant to the former. If the answer is no, the method proceeds to step 714, at which point a foamed latex or polymeric composition is applied to a tip portion of a condom former. If the answer is yes, the method 700 proceeds to step 708, where a coagulant is applied, such as a powder or coagulant solution. A decision is then made whether to dry the coagulant solution at step 710. If the answer is no, the method 700 proceeds to step 714. If the answer is yes, the method 700 proceeds to step 712 where the coagulant solution is dried at, for example, 65° C. for approximately 2-5 minutes, and the method 700 proceeds to step 714, as above.

The method 700 next proceeds to step 716, wherein the former is removed from the foamed latex or polymeric composition and dried at, for example, 70-110° C. for approximately 2-5 minutes, thereby forming a foam tip of the polymeric composition on the condom former. At step 718, a decision is made whether to apply a primer, as discussed more fully below, to the foam tip. If the answer is yes, the method 700 proceeds to step 720 and the condom former having the foam tip is dipped into a primer and/or coagulant solution, which may be deep enough to cover only the foam tip or, alternatively, cover most of the condom former as well as the foam tip. As above, a primer may be used to modify the surface chemistry to promote bonding between, for example, a first polymeric coating and a second polymeric coating. A coagulant may be used to de-stabilize the polymeric composition, promoting a gelling of the polymeric composition. If the answer is no, the method 700 proceeds directly to step 722, at which point the condom former having the foam tip is dipped into a bath of polymeric composition for a few to several seconds, wherein a polymeric layer, i.e., a tubular shaft, is adhered to the condom former and to the foam tip. The former having both the polymeric layer and the foam tip is then removed from the bath of polymeric composition.

At this point, a decision is made whether to make a condom having either a foam tip on one of an interior surface or an exterior surface or whether to make a condom having a foam tip on both an interior surface and an exterior surface. If a condom having a foam tip on an interior surface is to be made, the method 700 proceeds to step 724, at which point the polymeric layer is dried at, for example, 65° C. for approximately 2-5 minutes. At step 726, a beading step is performed. At step 728, the polymeric layer and the foam tip are cured at, for example, 90-120° C. for approximately 3-5 minutes, to form a condom having a foam tip. At step 730, the condom is optionally leached in hot water. At step 732, the method 700 ends. The condom having the foam tip may be rolled and packaged. Alternatively, the condom having the foam tip may be inverted, whereupon the foam tip is on the outside of the condom, and subsequently rolled and packaged.

If a condom having a foam tip on an interior surface and an exterior surface is to be made, the method 700 proceeds to step 740, at which point the condom former having the foam tip, which is on an interior surface of the tubular shaft, has a primer applied. The condom former is then dipped into a foamed latex composition at step 742 to form an exterior foam tip on the tubular shaft. At step 744, the tubular shaft having a foam tip on an interior surface and a foam tip on an exterior surface is dried, as discussed above. The method then returns to steps 726, 728, 730, and 732 as discussed above.

As used herein, the terms polymeric, elastomeric, thermoplastic elastomer, latex, and rubber are used interchangeably to describe material, such as a polymeric composition, used to form condoms in accordance with embodiments of the invention. Polymeric compositions include elastomeric compositions, polymeric compositions, latex compositions, and natural rubber compositions, synthetic compositions, and/or blends or mixtures thereof. The term “natural rubber latex” as used in this disclosure encompasses cured elastomeric material sourced from Hevea brasiliensis (the traditional rubber tree), non-Hevea rubber such as Parthenium argentatum (guayule), sunflower, goldenrod, and the like, as well as genetically modified variations of these or other biological sources. In some embodiments of the invention, condoms comprise the pre-vulcanized and post-vulcanized latex composition as disclosed in commonly-assigned U.S. Pat. No. 8,087,412, which is hereby incorporated by reference in its entirety. In some embodiments of the invention, synthetic polyisoprenes, carboxylated butadiene-nitriles, polyurethanes or polyurethane-polyurea copolymers, or combinations thereof are used.

The total solids content range of polymeric compositions, which may include a natural color or another color, range from about 28% to about 70%. At least one exemplary embodiment according to the invention comprises a polymeric composition having a total solids content of approximately 53% and may be foamed as discussed below. Moreover, processing aids, additives, rheological additives, stabilizers, and the like, known to those in the art, may be incorporated into any polymeric composition.

Furthermore, embodiments according to the invention comprise polymeric compositions having colorants and/or pigments, and further include glow-in-the-dark or fluorescent colorants or pigments. For example, at least one pigment according to embodiments of the present invention is a Quinacridone, such as Colanyl® Red E3B 130 manufactured by the Clariant Corp., or a Phthalocyanine, such as Colanyl® Blue A2R 131 also manufactured by the Clariant Corp., or combinations thereof. Examples of glow-in-the-dark pigments include photoluminescent pigments, such as SP-6-B distributed by Farben Technology but are not limited thereto.

Also, in some embodiments, one or more coagulant or primer solutions are disposed onto the condom, and in some embodiments, coagulant or primer solutions are not disposed onto the condom. Coagulant or primer solutions comprise concentration ranging from about 1% to about 50% by weight, and may include a natural color or another color. In some embodiments of the present invention, the coagulant concentration is about 5% by weight. According to some embodiments, the coagulant solution may contain Group I metal salts, Group II metal salts, or combinations thereof, and wetting agents ranging from 0.1-0.2% by weight in an aqueous or alcoholic solution. In some embodiments of the invention, the coagulant is an aqueous solution comprising 3.5% Calcium Nitrate and 96.5% water.

In at least one exemplary embodiment according to the invention, the coagulant solution comprises a 3-15% Calcium Nitrate or other Calcium salt, 2-10% Calcium Carbonate, and a small amount of surfactant and anti-foam agent, as are known to those in the art. Furthermore, at least one exemplary embodiment replaces Calcium Carbonate with a powder-free coagulant, such as a stearate, such as Calcium Stearate, so that post-washing steps may be omitted. Other suitable coagulants known to those in the art may also be used, such as calcium chloride, acetic acid, citric acid, and other strong and weak coagulants as are known to those in the art. Also, primer solutions comprise, for example, a 1-5% Calcium Nitrate or other Calcium salt, and a small amount of surfactant. In at least one embodiment according to the invention, the foamed composition(s) disclosed herein comprise a wetting agent. Foamed compositions, as in the methods 500 and 600, having a wetting agent can optionally omit the step of using a primer.

Embodiments of the invention comprise foamed compositions compounded using the following process steps. Any conventional natural rubber, synthetic polyisoprene, nitrile latex, polyurethane latex, may be used as a polymeric resin. The polymeric resin is compounded, optionally with a colorant or pigment, with a foaming agent and other additives to stabilize the foam, as is known to those in the art. The polymeric resin and the foaming agent and optional additives are stirred with a high shear mixer or whisker at a high speed, e.g., approximately 500 RPM, which is maintained until an open cell foam of a desired air content is achieved, which is generally 100% of the originally compounded volume. The viscosity of the foamed composition is adjusted, and the foam is refined by reducing the mixing speed to approximately 200-300 RPM, which produces a bubble/foam size of approximately 5-50 microns. The size of the foam can be varied to produce a desired foam layer thickness on the condom. A smaller foam size structure results in greater semen absorption. For example, at least one foamed tip having a thickness of 25 microns, according to embodiments of the invention, can absorb, for example, 0.5-0.6 mL of water or silicone oil. Alternatively, the foam may be refined using an appropriate whipping impeller at an appropriate speed and refining an air bubble size at a reduced speed to form an open-cell foam as is disclosed in commonly assigned U.S. Pat. No. 8,640,504, which is herein incorporated by reference in its entirety.

The methods 500 and/or 600 may also be used to form additional polymeric articles having foam tips. For example, a surgical examination glove may have foam tips at one or more fingers/thumb, on either or both of the interior of finger stalls and/or on an external surface thereof. Any of the methods and/or processes disclosed and described herein may be used to manufacture supported and/or unsupported gloves. Also, a supported glove, i.e., a fabric liner may have a foam disposed on the tips of the fingers and thumb of the fabric liner and a subsequent layer of unfoamed polymeric composition applied to all or parts of the fabric liner, irrespective of whether coated with a foamed composition.

While the foregoing is directed to embodiments of the invention, other embodiments of the invention may be devised without departing from the scope thereof, and the scope thereof is determined by the following claims.

What is claimed is:

1. A condom, comprising:
a tubular shaft comprising a polymeric composition and having an open end and a closed end having a tip;
a hollow interior chamber disposed between the open end and the closed end; and
an open-cell foam tip portion formed on at least one of an inner surface or an outer surface of the tip,
wherein the tubular shaft and the open end are adapted for receiving a penis of a wearer.

2. The condom of claim 1, wherein the open-cell foam tip portion is adapted to deliver at least one active ingredient to the penis of the wearer.

3. The condom of claim 1, wherein the open-cell foam tip portion is adapted to localize the at least one active ingredient.

4. The condom of claim 1, wherein the open-cell foam tip portion is integrally formed with the tubular shaft.

5. The condom of claim 2, wherein the at least one active ingredient is an anti-viral, an anti-microbial, an anti-fungal, a vasodilator, a benzocaine, a warming liquid or gel, a spermicide, or a lubricant.

6. The condom of claim 1, wherein the open-cell foam tip portion is approximately 20-60 mm in length.

7. The condom of claim 1, wherein the open-cell foam tip portion comprises a cross-sectional thickness of approximately 20-100 microns.

8. The condom of claim 1, wherein the open cell polymeric foam comprises at least one of natural polyisoprenes, synthetic polyisoprenes, carboxylated butadiene-nitriles, polyurethanes or polyurethane-polyurea copolymers, or combinations thereof.

9. The condom of claim 1, wherein the open cell polymeric foam has a cell size of approximately 5-50 microns.

10. The condom of claim 1, wherein the tip portion is an open cell foam tip formed on an interior surface of the hollow interior chamber.

11. A method for forming a condom, comprising:
dipping a condom into a foamed latex composition, thereby coating a layer of foamed latex composition onto the condom; and
curing the layer of foamed latex composition to form a condom, wherein the layer of foamed latex composition is an open cell polymeric foam.

12. The method for forming a condom of claim 11, wherein the open cell polymeric foam comprises at least one of natural polyisoprenes, synthetic polyisoprenes, carboxylated butadiene-nitriles, polyurethanes or polyurethane-polyurea copolymers, or combinations thereof.

13. The method for forming a condom of claim 11, wherein the open cell foam has a cell size of approximately 5-50 microns.

14. A method for forming a condom, comprising:
disposing a polymeric composition onto a former, thereby forming a first polymeric layer;
removing the former from the polymeric composition;
disposing a foamed polymeric composition onto the first polymeric layer, thereby forming a foamed second polymeric layer; and
curing the first polymeric layer and the foamed second polymeric layer to form a condom.

15. The method for forming a condom of claim 14, wherein the foamed second polymeric foam comprises at least one of natural polyisoprenes, synthetic polyisoprenes, carboxylated butadiene-nitriles, polyurethanes or polyurethane-polyurea copolymers, or combinations thereof.

16. The method for forming a condom of claim 14, further including disposing at least one active ingredient into the foamed second polymeric layer.

17. The method of claim 16, wherein the at least one active ingredient is an anti-viral, an anti-microbial, an anti-fungal, a vasodilator a benzocaine, a warming liquid or gel, a spermicide, or a lubricant.

18. The method for forming a condom of claim 14, wherein the first polymeric layer comprises at least one of natural polyisoprenes, synthetic polyisoprenes, carboxylated butadiene-nitriles, polyurethanes or polyurethane-polyurea copolymers, or combinations thereof.

19. The method for forming a condom of claim 14, further comprising disposing a coagulant onto the first polymeric layer, wherein the coagulant is disposed before the disposing the foamed polymeric composition onto the first polymeric layer.

20. The method for forming a condom of claim 14, further comprising a step for inverting the condom.
